(19) United States
(12) Patent Application Publication
(10) Pub. No.: US 2021/0018507 A1
(43) Pub. Date: Jan. 21, 2021

(54) SALL.IVARY PROTEIN BIOMARKERS FOR THE DIAGNOSIS AND PROGNOSIS OF HEAD AND NECK CANCERS, AND PRECANCERS

(71) Applicants: Mazumdar Shaw Medical Foundation, Bangalore (IN); Narayana Hrudayalaya Foundation, Bangalore (IN); Institute of Bioinformatics, Bangalore (IN); Department of Biotechnology, Ministry of Science and Technology, Gevernment of India, New Delhi (IN)

(72) Inventors: Moni Abraham Kuriakose, Bangalore (IN); Ravi Sirdeshmukh, Bangalore (IN); Amritha Suresh, Bangalore (IN); Priya Sivadasan, Bangalore (IN); Manoj Kumar Gupta, Bangalore (IN)

(21) Appl. No.: 16/978,551

(22) PCT Filed: Mar. 4, 2018
(86) PCT No.: PCT/IB2018/051375

§ 371 Date: Sep. 4, 2020

(51) Int. Cl. G01N 33/574

(52) U.S. Cl. CPC G01N 33/57488; G01N 33/57407

(57) ABSTRACT

The present disclosure relates to a panel of biomarkers for the detection/diagnosis and prognosis of head and neck squamous cell carcinoma (HNSCC). Biomarkers for the detection/diagnosis of carcinoma cells identified by proteomic profiling and, detection/identification of selected proteins that are differentially regulated, in a biological sample from an individual as candidate markers. The biomarkers are useful for non-invasive early detection/prognosis focusing on the protein profiling of saliva at different stages of oral pre-cancer and cancer progression. Furthermore, the present invention deals with novel methods of diagnosing and for providing a prognosis for oral cancer and periodontal disease. In addition, the invention also provides kits that are useful for the practice of the methods of the invention.

### TECHNICAL FIELD OF THE INVENTION

The present invention is in the technical field of biomarkers for head-and-neck cancers, including oral cancers. In particular, the invention relates to, sensitive, specific and reliable detection and identification of salivary biomarkers that are specifically produced in head and neck squamous cell carcinoma (HNSCC).

### BACKGROUND OF THE INVENTION

Head and neck squamous cell carcinomas (HNSCC) are one of the most prevalent cancers worldwide, with specifically high incidence in the Asian sub-continent. Oral cancers constitute one of the most common types of head and neck cancers and despite easy accessibility of oral cavity for visual examination; these cancers are typically detected in their advanced stages.

In India, 60-80% of HNSCC present are with advanced disease and poor overall survival. These statistics point out to the need for an increased focus on early detection as a strategy for downstaging of the disease at presentation [1].

A study from India demonstrated that visual screening by trained health workers can lower mortality of the disease, especially in individuals with a history of tobacco use [2]. However, visual screening is skill-dependent, and may not detect occult disease.

In the present scenario, the additional challenge associated with oral cancer is accurate prognosis subsequent to treatment. In patients with oral cancer, surgery is the preferred mode of treatment. Despite great progress in chemotherapy, radiotherapy, and targeted therapy in the last three decades, the prognosis of OSCC remains poor due to drug resistance, aggressive local invasion and metastasis, leading to recurrence. 26-47% of patients are known to develop a recurrence within 2 years of surgical resection with an annual 5% chance of developing a second primary tumor [3].

Periodic monitoring of patients for disease progression can effectively help in early intervention and enable improved survival rates [4].

At present, clinical, histopathological, radiological examinations and tissue biopsy with histological assessment are the gold standards for detection of high risk lesions and metastasis. However, their applications in community cancer screening program/post-treatment surveillance are arguable due to their invasive nature. In addition, this technique needs a trained health-care provider, and is considered invasive, painful, expensive and time consuming. Recent clinical diagnostic tools for early detection of oral cancer also include tolonium chloride or toluidine blue dye, Oral CDx brush biopsy kits, and optical imaging systems. These methods have issues with sensitivity and specificity with some of them being highly sensitive but with poor specificity. Although oral exfoliative cytology and brush biopsy techniques are helpful in establishing a more definitive diagnosis of already visible lesions, they are of no value in detecting mucosal changes that are not readily visible to the naked eye. Currently, the new innovative visual-based techniques show promising results, but lack strong evidence to support their effectiveness in early detection. On the other hand, prognosis of the patients is currently predicted based on clinical or pathological staging and/or based on imaging (CT/PET-SCAN). These methods are either subjective or lack accuracy in detecting micro-metastatic deposits leading to inappropriate patient management often resulting in an unfavorable outcome.

In addition, histological diagnosis is also found wanting an accurate risk prediction, especially in the case of pre-malignant lesions, necessitating the exploration of molecular marker-based methods.

Saliva as a diagnostic medium offers an easy, inexpensive, safe, and non-invasive approach. [5] It is one of the most complex, versatile, and important body fluids, which reflects a large range of physiological needs and information. Studies have shown correlation of DNA based alterations and levels of salivary proteins/mRNAs to be associated with OSCC and lung cancers [6-8]. Several studies have established the potential utility of salivary biomarkers in disease diagnostics [9]. Researchers have also reported that proteome/genome-wide approach can be employed towards the identification and validation of disease biomarkers in saliva.

Early diagnosis of oral cancers is important, as successful treatment of cancers is very much dependent on early detection.

Early oral cancerous lesions rarely show distinct clinical characteristics. Furthermore, there are growing body of cases that shows some premalignant and early cancerous lesions cannot be not clearly diagnosed by visual inspection.

At present, there is a need for cataloging of molecular changes that are known to precede clinical and histological manifestations in the body fluids like saliva as an effective strategy towards developing easy, accurate and non-invasive methods for early detection/screening and for disease surveillance.

In addition, there is a need for integration of early detection/prognosis and screening of oral cancer based on protein biomarkers, along with conventional oral examination.

Furthermore, there is also a need for highly specific marker based cost-effective oral cancer screening method as a detection strategy that can be implemented in high-risk populations.

In summary, there is an urgent need in the art to develop a highly specific marker based cost-effective oral cancer screening methods for early detection/prognosis of oral cancer.

### SUMMARY OF THE INVENTION

The primary objective of the present invention is to provide a panel of salivary biomarkers for the detection/diagnosis of head and neck squamous cell carcinoma (HNSCC). Proteomic profiling has identified a list of 93 upregulated proteins as candidate markers for early detection wherein the oral cancer biomarker can be directly detected in the specimen of the body fluid of a subject and thus can provide effective clinical diagnosis of oral cancer.

In one of the embodiment, the present invention provides biomarkers for the detection/diagnosis of carcinoma cells by proteomic profiling, detection and identification of proteins selected from those found in any one of Table 1 comprising 93 differentially regulated proteins or Table 2 comprising 179 differentially regulated proteins, in a biological sample from an individual as candidate markers for early detection, detecting premalignant lesion and tumors marked by distinct high level in leukoplakia, that can differentiate lymph node negative tumor from lymph node positive tumors, salivary biomarkers that have high potential for use in early diagnosis of dysplastic lesions/cancers of the oral cavity, useful for early detection of carcinoma cells and more preferably the biomarkers that are useful for non-invasive early detection/prognosis focusing on the proteomic profiling of saliva at different stages of oral cancer progression.

According to a further aspect of the present invention, the biomarker for diagnosis carcinoma cells, wherein said biomarkers are salivary biomarkers.

In preferred embodiments, the biomarker for diagnosis of carcinoma cells, wherein said carcinoma/cancer is oral carcinomas and other sites of head and neck squamous cell carcinoma (HNSCC).

In preferred embodiments, 8 representative markers; S100A7, CD44, COL5A1 and S100P comprise a related group of molecules with distinct high level in leukoplakia (premalignant lesion) and tumor; and COL1A1, CD44, S100A11 and a1AT comprise a related group of molecules that can differentiate lymph node positive tumor from lymph node negativepositive tumors, suggesting high potential for use in early diagnosis of dysplastic lesions/cancers of the oral cavity, that are detectable in saliva, and are found to be useful for early detection of HNSCC. In particular, the biomarkers are useful for non-invasive early detection/prognosis focusing on the proteomic profiling of saliva at different stages of oral cancer progression.

According to a further exemplary aspect of the present invention, it provides a method of detecting HNSCC in a subject, through the presence of one or more of the biomarkers in a subject sample, and further correlating their level with increased risk of developing HNSCC. Subject samples are compared to the level of occurrence of the biomarker(s) in normal subjects, wherein the biomarkers are selected from S100A7, CD44, COL5A1, S100P or other markers selected from the subset of Table 1 comprising 93 differentially regulated proteins or Table 2 comprising 179 differentially regulated proteins.

Thus, in one particular embodiment, increased COL1A1, CD44, S100A11, a1AT or other markers that will be validated from the subset of 93 candidate markers, in saliva and/or other biological samples are indicative of tumor stage. For example, low levels are indicative of early stage cancer; and higher levels are indicative of later stage HNSCC.

In yet another embodiment, a method of predicting the course of HNSCC in a subject is provided, comprising measurement of one or more of S100A7, CD44, COL5A1, S100P, COL1A1, CD44, S100A11, a1AT levels in a biological sample obtained from said subject, wherein the degree of increase of S100A7, CD44, COL5A1, S100P, COL1A1, CD44, S100A11, a1AT is indicative of risk of recurrent disease or of more severe disease of HNSCC.

According to a further exemplary aspect of the present invention, a method of detecting oral carcinomas, preferably comprises of comparison of levels of salivary biomarkers S100A7, CD44, COL5A1, S100P, COL1A1, CD44, S100A11, a1AT or other markers from the subset of 93 preferred biomarkers from Table A or 179 preferred biomarkers from Table B, identified with those present in normal (without the presence of pre/existing cancer stage) and also evaluating the levels of such biomarkers indicative of the different stages of carcinoma/cancer such as pre, early or advanced.

In yet another embodiment, a scoring pipeline has been developed encompassing multiple parameters such as technical quality, previous identifications, association with cancer in general, association with oral cancer and secretability for evaluating 93 proteins from Table A or 179 proteins from Table B, such biomarkers by performing ELISA and/or immunoassays can assess their preferable association with cancers of the oral cavity. This pipeline enables identification of markers with high confidence that can be prioritized for validation.

According to a further exemplary aspect of the present invention, a method of identifying, diagnosing or providing a prognosis for oral cancer in a biological sample from a subject, the method comprising the steps of:
(a) detecting a presence or level of one or more biomarker in a biological sample from a subject, wherein the one or more biomarker is selected from those found in any one of Tables A or B in a biological sample from an individual;
(b) and Identifying/determining whether or not said biomarker is differentially expressed in the sample, thereby diagnosing or providing a prognosis for oral cancer.

According to a further exemplary aspect of the present invention, the biomarkers can be detected, for example, using an immunoassay, a protein assay or binding assay. The higher level of biomarkers in a subject sample as compared to reference normal suggests the occurrence of HNSCC.

In yet another embodiment, preferably, the sample is saliva for the evaluation of these markers. However, other samples can be selected from body fluid, wherein said body fluid is plasma, serum, urine, peripheral blood, sputum, saliva, bone marrow, pleural and peritoneal fluid, or mucosal secretion.

According to a further exemplary aspect of the present invention, non-limiting examples of use of salivary biomarkers in the detection of head and neck cancers including oral cancers, cancers of larynx, pharynx, primary or secondary cancers as a cost-effective method for screening on large scale, for prognosis, screening at various stages of cancers in the subjects during and post -treatment, in the form of kits, strips, swabs, sticks, discs, meters and such other easily usable, disposable, multi-utility kits for measuring the preferred biomarkers to detect head and neck cancers including oral cancers with specificity, sensitivity, accuracy, speed, reliability and reproducibility.

In yet another embodiment, a kit for use in diagnosing or providing a prognosis for oral cancer in a biological sample from a subject, the kit comprising at least one reagent that can detect the cancer biomarker, wherein at least one oral cancer biomarker is selected from the group consisting of those found in Table 1 and Table 2.

In yet another embodiment, an antibody generated against an epitope selected from the group consisting of those found in Table 1 and Table 2.

In summary, the present invention deals with a panel of biomarkers for the detection/diagnosis of carcinoma cells identified by proteomic profiling, detection and identification of proteins selected from those found in any one of Table 1 comprising 93 differentially regulated proteins or Table 2 comprising 179 differentially regulated proteins, in a biological sample from an individual as candidate markers for early detection, detecting premalignant lesion and tumors marked by distinct high level in leukoplakia, that can differentiate lymph node negative tumor from lymph node positive tumors, salivary biomarkers that have high potential for use in early diagnosis of dysplastic lesions/cancers of the oral cavity, useful for early detection of carcinoma cells and more preferably the biomarkers that are useful for non-invasive early detection/prognosis focusing on the proteomic profiling of saliva at different stages of oral cancer progression. Finally, kits are provided that find use in the practice of the methods of the invention, wherein the kit comprising of at least one reagent that specifically binds to a cancer biomarker.

Several aspects of the invention are described below with reference to examples for illustration. However, one skilled in the relevant art will recognize that the invention can be practiced without one or more of the specific details or with other methods, components, materials and so forth. In other instances, well-known structures, materials, or operations are not shown in detail to avoid obscuring the features of the invention. Furthermore, the features/aspects described can be practiced in various combinations, though only some of the combinations are described herein for conciseness.

As will be appreciated by a person skilled in the art the present invention provides a variety of following advantages.

The present invention deals with a panel of biomarkers for the detection/diagnosis and prognosis of head and neck squamous cell carcinoma (HNSCC).

The present invention deals with a panel of 8 representative markers; S100A7, CD44, COL5A1 and S100P comprise a related group of molecules with distinct high level in early stage leukoplakia and tumor; and COL1A1, CD44, S100A11 and a1AT comprise a related group of molecules that can differentiate lymph node negative tumor from lymph node positive tumors. These results suggest high potential for use of these markers that are detectable in saliva, in early diagnosis of dysplastic lesions/cancers of the oral cavity and for detection of nodal metastasis.

In particular, the present invention describes that the biomarkers identified by the proteomic profiling of saliva at different stages of oral cancer progression are useful for non-invasive early detection/prognosis

The biomarkers can be detected, for example, using an immunoassay, a protein assay or binding assay or a microfluidics assay. The elevated level of biomarkers and/or total protein in a subject sample as compared to values in normal healthy controls suggests the susceptibility to or occurrence of HNSCC.

The subject sample may be selected, for example, from the sample group consisting of oral rinse, saliva, blood plasma, serum, urine, tissue, blood and cells, subject to further validations with these samples. Preferably, the sample is saliva.

In addition to the markers listed, further candidates can also be selected from the 93 high confidence markers to evaluate their clinical applicability in early detection/prognosis.

The scoring pipeline developed incorporating the technical and functional aspects of all the proteins enabled identification of top candidates that can be prioritized for validation.

The advantages of this invention include but not limited to,

Individual or combination of markers can be used to develop assay systems for the early diagnosis of high risk lesions.

Individual or combination of markers can be used for community based screening of high-risk populations to identify patients at risk for developing oral premalignant lesion and/or cancer.

Individual or combination of markers can be used to develop monitoring or surveillance systems to monitor disease progression.

Individual or combination of markers can be used to predict the development of nodal metastasis.

Scoring pipeline to identify the markers of high technical and functional relevance for further validations

The marker panel can be used for the development of a Point-of-Care assay system that can be applied towards early detection, screening, and disease progression.

These assay systems can be used for patients susceptible to or diagnosed with oral cancer and the subsites of head and neck cancer such as cancers of the larynx and pharynx.

### BRIEF DESCRIPTION OF THE DRAWINGS

Example embodiments of the present invention will be described with reference to the accompanying drawings briefly described below.

FIG. 1 illustrates the need for non-invasive biomarkers in early detection of oral cancer.

FIG. 2 illustrates a schematic representation of detection of salivary Biomarkers in the early stage of cancer.

FIG. 3 illustrates the experimental design for the identification of biomarkers in Oral cancer.

FIG. 4 illustrates the analytical Pipeline for selection of significant salivary biomarkers.

FIG. 5 illustrates the CD44, COL5A1, S 100P, S100A7 protein levels in the saliva of premalignant and malignant lesions of the oral cavity. Box plots indicate the average concentrations of CD44 (A), COL5A1 (B), S100P (C) and S100A7 (D) in the saliva of dysplastic leukoplakia and OSCC. The oral cancer samples include lymph node negative and lymph node positive OSCC.

FIG. 6 (E, F, G and H) illustrates the COL1A1, a1AT, S100A11, S100A15 protein levels in the saliva of premalignant and malignant lesions of the oral cavity. Box plots indicate the average concentrations of COL1A1 (E), a1AT (F), S100A11 (G) and S100A15 (H) in the saliva of dysplastic leukoplakia and OSCC. The oral cancer samples include lymph node negative and lymph node positive OSCC.

FIG. 7 (A, B and C) illustrates the significance of the three markers (7A) S100 A7, (7B) S100P and (7C) CD44 in early detection of oral premalignant disorders in the saliva of Normal, leukoplakia and OSCC patients. Box plots indicate the Mean concentrations in each cohort and the ROC curve analysis indicates the specificity and sensitivity of the markers.

FIG. 8 (A, B and C) Shows the ROC analysis of the three early detection markers (8A) S100 A7, (8B) S100P and (8C) CD44 Salivary markers for the detection of Oral premalignant lesions and cancers.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

As used herein, the singular forms “a”, “an”, and “the” include both singular and plural referents unless the context clearly dictates otherwise. By way of example, “a dosage” refers to one or more than one dosage.

The terms “comprising”, “comprises” and “comprised of” as used herein are synonymous with “including”, “includes” or “containing”, “contains”, and are inclusive or open-ended and do not exclude additional, non-recited members, elements or method steps.

All documents cited in the present specification are hereby incorporated by reference in their totality. In particular, the teachings of all documents herein specifically referred to are incorporated by reference.

Example embodiments of the present invention are described with reference to the accompanying figures.

In the drawings, like reference numbers generally indicate identical, functionally similar, and/or structurally similar elements. The drawing in which an element first appears is indicated by the leftmost digit(s) in the corresponding reference number.

### Definitions

The following terms are used as defined below throughout this application unless otherwise indicated.

The terms “tumour” or “tumour tissue” refer to an abnormal mass of tissue which results from uncontrolled cell division. A tumour or tumour tissue comprises “tumour cells” which are neoplastic cells with anomalous growth properties and no functional bodily function. Tumours, tumour cells and tumour tissue can be benign or malignant.

“Marker” or “biomarker” are used interchangeably, and in the context of the present invention refer to a polypeptide, which is differentially present in a sample collected from patients having HNSCC as compared to a comparable sample taken from control subjects.

The phrase “differentially present” refers to differences in the quantity of the marker present in a sample taken from patients as compared to a control subject. A biomarker can be differentially present in terms of frequency, quantity or both.

“Diagnostic” means identifying a pathologic condition.

The terms “detection”, “detecting” and the like, may be used in the context of detecting markers or biomarkers.

A “test amount” of a marker refers to an amount of a marker present in a sample being tested. A test amount can be either in absolute amount (e.g., μg/ml) or a relative amount (e.g., relative intensity of signals).

The terms “polypeptide,” “peptide” and “protein” are used interchangeably herein to refer to a polymer of amino acid residues. “Polypeptide,” “peptide” and “protein” can be modified, e.g., by the addition of carbohydrate residues to form glycoproteins.

“Detectable moiety” or a “label” refers to spectroscopic, photochemical, biochemical, immunochemical, or chemical means of detection of a composition. For example, labels may include ³²P, ³⁵S, fluorescent dyes, biotin-streptavidin, dioxigenin, haptens, electron-dense reagents, and enzymes. The detectable moiety generates a measurable signal that can quantify the amount of bound detectable moiety in a sample. Quantitation of the signal is done by scintillation counting, densitometry, or flow cytometry.

“Antibody” refers to a polypeptide ligand encoded by an immunoglobulin gene(s), which specifically binds and recognizes an epitope.

The terms “subject”, “patient” or “individual” generally refer to a human or mammals.

“Sample” refers to a polynucleotides, antibodies fragments, polypeptides, peptides, genomic DNA, RNA, or cDNA, polypeptides, a cell, a tissue, and derivatives thereof may comprise a bodily fluid or a soluble cell preparation, or culture media, a chromosome, an organelle, or membrane isolated or extracted from a cell.

Subject refers to a subject or patient can include, but is not limited to, mammals such as bovine, avian, ovine, porcine, canine, equine, feline, or primate animals (including humans and non-human primates).

The subject can have a pre-existing disease or condition, such as cancer. Furthermore, the subject may not have any known pre-existing condition. In addition, the subject may also be non-responsive to an existing or past treatment, such as a treatment for cancer.

“Body fluid” refers to, but is not limited to, plasma, serum, urine, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood.

The term “oral cancer” refers to a group of malignant or neoplastic cancers originating in the oral cavity of an individual. Non-limiting examples of oral cancers include cancers of the buccal vestibule, hard or soft palate, tongue, gums (including gingival and alveolar carcinomas), lingual cancer, buccal mucosa carcinoma, and the like.

“Head and neck squamous cell carcinoma” refers to group of cancers of epithelial cell origin originating in the head and neck, these tumors may arise from diverse locations, including the oral cavity, oropharynx, hypopharynx, larynx, and nasopharynx. The oral cavity includes the buccal mucosa, upper and lower alveolar ridges, floor of the mouth, retromolar trigone, hard palate, and anterior two thirds of the tongue.

“Periodontal disease” refers diseases affecting the gums of an individual, including gingivitis, periodontitis, and the like.

“Therapeutically effective amount or dose” refers to a dose that produces effects for which it is administered. The exact dose depends on the purpose of the treatment.

“Metastasis” refers to spread of a cancer from the primary origin to other tissues and parts of the body, such as the lymph nodes.

“Saliva” refers to any watery discharge from the mouth.

“Prognosis” refers to prediction of the likelihood of metastasis, predictions of disease free and overall survival, the probable course and outcome of cancer therapy, or the likelihood of recovery from the cancer, in a subject.

“Diagnosis” refers to identification of a disease state, such as cancer in a subject. The methods of diagnosis provided by the present invention can be combined with other methods of diagnosis well known in the art. Non-limiting examples of other methods of diagnosis include, detection of known disease biomarkers in saliva samples, co-axial tomography (CAT) scans, positron emission tomography (PET), oral radiography, oral biopsy, radionuclide scanning, and the like.

“Nucleic acid” refers to deoxyribonucleotides or ribonucleotides and polymers thereof in either single- or double-stranded form, and complements thereof

A particular nucleic acid sequence may also implicitly encompass conservatively modified variants thereof (e.g., degenerate codon substitutions) and complementary sequences, in addition to the sequence explicitly indicated. Furthermore, degenerate codon substitutions may be achieved by generating sequences in which the third position of one or more selected (or all) codons is substituted with mixed-base and/or deoxyinosine residues.

The cancer characterized by the methods of the invention can comprise, without limitation, a carcinoma, a germ cell tumor, a blastoma, a sarcoma, a lymphoma or leukemia, or other cancers. Carcinomas include without limitation transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, epithelial neoplasms, squamous cell neoplasms squamous cell carcinoma, basal cell neoplasms basal cell carcinoma, prolactinoma, oncocytoma, hurthle cell adenoma, renal cell carcinoma, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, Sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, chondrosarcoma, Ewing's sarcoma, malignant hemangio endothelioma, fibrosarcoma, hemangiopericytoma, hemangiosarcoma, kaposi's sarcoma, leiomyosarcoma, liposarcoma, lymphangiosarcoma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as Waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, T cell large granular lymphocytic leukemia, aggressive NK cell leukemia, adult T cell leukemia/lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, polyembryoma, and gonadoblastoma. Blastoma includes without limitation nephroblastoma, medulloblastoma, nongerminomatous germ cell tumor, embryonal carcinoma, endodermal sinus tumor, choriocarcinoma, teratoma, and retinoblastoma. Other cancers include without limitation labial carcinoma, adenocarcinoma, larynx carcinoma, hypopharynx carcinoma, tongue carcinoma, salivary gland carcinoma, gastric carcinoma, thyroid cancer (medullary and papillary thyroid carcinoma), renal carcinoma, kidney parenchyma carcinoma, cervix carcinoma, uterine corpus carcinoma, meningioma, endometrium carcinoma, chorion carcinoma, testis carcinoma, urinary carcinoma, melanoma, brain tumors such as glioblastoma, astrocytoma, medulloblastoma and peripheral neuroectodermal tumors, gall bladder carcinoma, bronchial carcinoma, multiple myeloma, basalioma, teratoma, osteosarcoma, chondrosarcoma, retinoblastoma, choroidea melanoma, seminoma, rhabdomyosarcoma, craniopharyngeoma, fibrosarcoma, Ewing sarcoma, myosarcoma, liposarcoma, and plasmocytoma.

### Diagnostic and Prognostic Methods

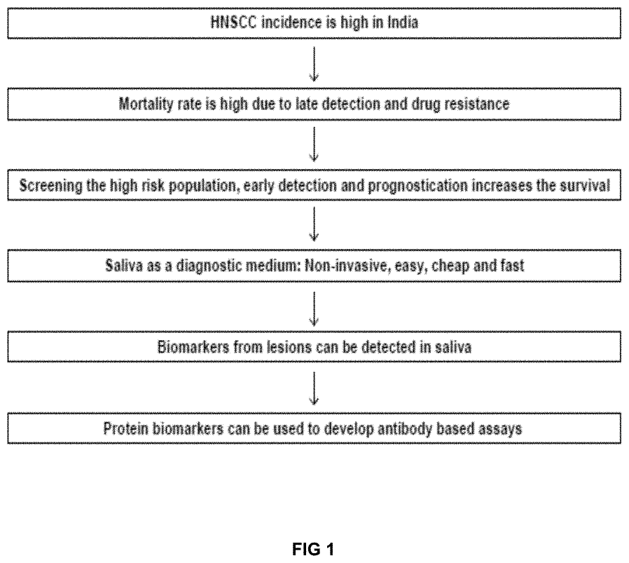

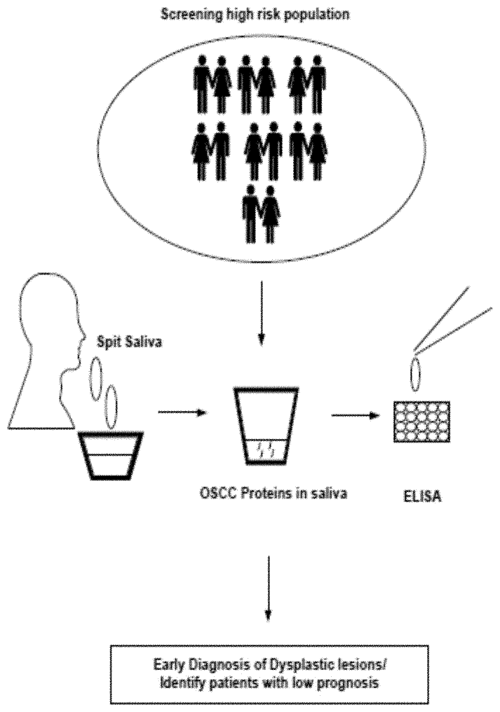

Inventors addressed the needs of non-invasive early detection/prognosis focusing on the proteomic profiling of saliva at different stages of oral cancer progression (FIG. 1 and FIG. 2). The present disclosure relates to the present invention discloses a panel of biomarkers (n=93) for the detection/diagnosis of head and neck squamous cell carcinoma (HNSCC).

More particularly, 8 representative markers; S100A7, CD44, COL5A1 and S100P comprise a related group of molecules with distinct high level in early stage leukoplakia and tumor; and COL1A1, CD44, S100A11 and a1AT comprise a related group of molecules that can differentiate lymph node negative tumor from lymph node positive tumors, with high potential for use in early diagnosis of dysplastic lesions/cancers of the oral cavity, that are detectable in saliva, and/or are found to be useful for early detection of nodal metastasis in HNSCC. In particular, the biomarkers are useful for non-invasive early detection/prognosis focusing on the proteomic profile of saliva at different stages of oral cancer progression.

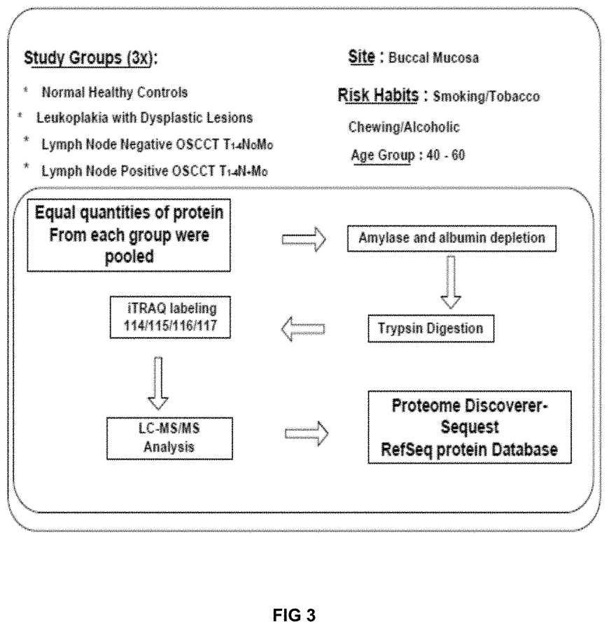

Early diagnosis and prognosis using saliva based molecular markers was attempted as a non-invasive solution for down-staging the disease and improving outcome. Proteomic profiling was carried out from the saliva of well-annotated patient samples and subsequently subsets of the highly significant molecules were validated by ELISA in an independent cohort of samples. The experimental pipeline is provided in FIG. 3.

The proteomic profiling and subsequent analysis was carried out in leukoplakia patients with dysplastic lesions, OSCC patients who were lymph node negative (NO; n=15) and those who were lymph node positive and healthy controls. The cell free saliva were analyzed using iTRAQ based quantitative proteomic analysis We identified a total of 1319 proteins from triplicate experiments, 179 of them were found with altered levels from various paired comparisons, 93 being up-regulated (≥1.5 fold). A scoring system was made for these 93 proteins based on their tumor and biological relevance and secretability assessed using prediction tools (Exocarta, Signal P and Secretome P). Thirty proteins were thus shortlisted that included members between any two or all three consecutive stages—leukoplakia, N0 and N+. The annotated list of 30 priority proteins along with their proteotypic peptides is provided as a resource for targeted investigations and development of clinical applications. We verified 8 representative molecules (S100A7, CD44, COL5A1, COL1A1, S100A11, S100A15, a1AT and S100P) by ELISA in independent cohorts of patients. S100A7, CD44, COL5A1 and S100P levels were high in early stage leukoplakia and tumor; COL1A1, CD44, S100A11 and a1AT can differentiate lymph node negative tumor from lymph node positive tumors, suggesting high potential for use in early diagnosis of dysplastic lesions/cancers of the oral cavity. The study shows that salivary proteomics and protein markers to be a promising approach to develop saliva based diagnostic methods and technologies. We found that S100A7, CD44, COL5A1 and S100P as promising markers for early diagnosis of oral cancer (TABLE 1 AND TABLE 2).

### METHODOLOGY

### Proteomic Profiling for Identification of Biomarkers:

The study subjects were leukoplakia patients with dysplastic lesions (n=15), lymph node negative (N0; n=15) and lymph node positive (N+; n=15) patients with carcinoma of buccal mucosa and healthy controls (n=15). The cell free saliva from 5 patients of each group were pooled, and the pools were analyzed using iTRAQ based quantitative proteomic analysis on Orbitrap Velos high resolution mass spectrometer. The protein identifications from all the 3 experiments when pooled resulted in total of 1319 proteins. In order to select differential proteins concordant between the three experiments, quality control of the data was carried out.

Quality control of the data retrieved from proteome discoverer was carried out at two levels i.e., at peptide level and protein level.

At peptide level, proteins with two or more PSMs were selected and the Coefficient of variation was calculated for the fold differences. Proteins with more than 40% variability in fold change at the PSM levels were removed from each one of the disease conditions in each experiment.

Proteins which are identified in all three or any two of the triplicate experiments and passed the QC at the peptide levels were further considered for QC at the protein level. Proteins with less than 40% variability in the fold change between the 3 or 2 experiments for each disease condition were selected for further analysis. Proteins with ≥ or ≤1.5 fold were considered as differentials.

Data Analysis: Gene ontology analysis was carried out to identify the biological process and molecular function of these proteins. These differential proteins were further classified based on secretory potential using Signal P, Secretome P, and Exocarta. The data were also compared to the protein list in the OSCC database (developed in our lab) in order to assess its presence in OSCC tissue and also to understand the chromosomal locus. These proteins were also checked in Human Protein Atlas (HPA) for their expression levels in different tissues and in cancers. In addition, the proteins in saliva, which were commonly expressed in both leukoplakia and oral cancer (N0 or N+) were also separated. A scoring system was created based on technical confidence (identification in the replicate studies, peptide numbers), secretory potential, and association with cancer/expression level in cancer tissues. Each protein was scored based on these criteria and the best markers were selected based on the score for further validation.

### Results:

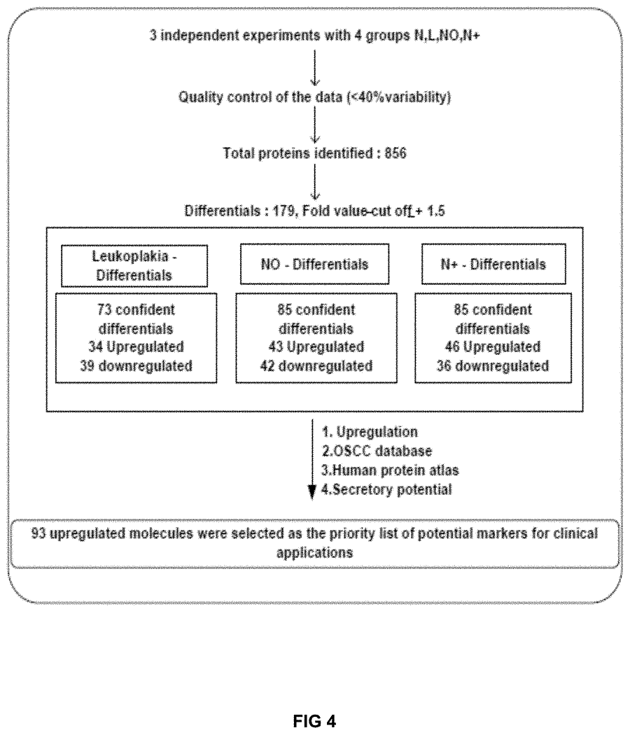

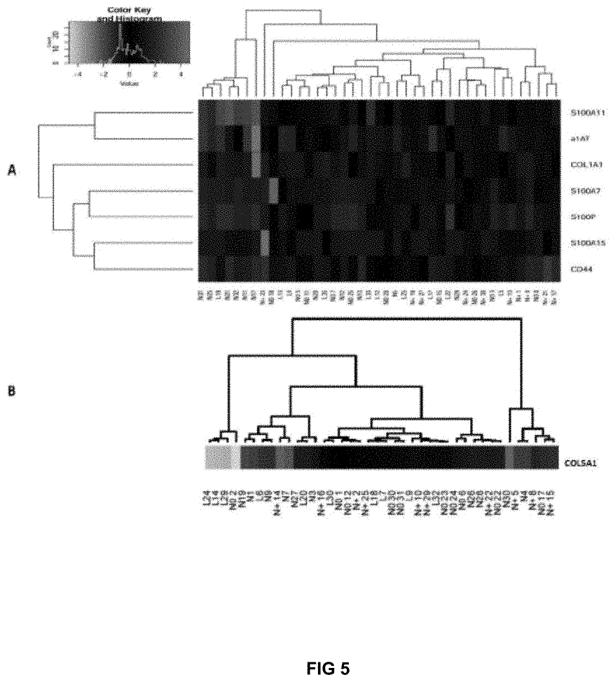

The protein identifications from all the 3 experiments when pooled, resulted in total of 1319 proteins. In order to select differential proteins concordant between the three experiments, quality control of the data was carried out as mentioned earlier. This resulted in the identification of 856 proteins from 5556 peptides; 179 proteins were found as differentially expressed when combined from various paired comparisons. Assessment between the different patient subgroups revealed that when compared to the normal, 73 proteins were identified as differentials in leukoplakia, 85 in lymph node-negative cancer and 85 in lymph node-positive cancers (FIG. 4).

TABLE 1
179 differentials expressed proteins

| | | Protein | Gene | |
|---|---|---|---|---|
| Sl No | Accession | accession | Symbol | Gene Description |
| 1 | 21071030 | NP_570602.2 | A1BG | alpha-1B-glycoprotein precursor |
| 2 | 321400142 | NP_001189486.1 | CD44 | CD44 antigen isoform 8 precursor |
| 3 | 4501881 | NP_001091.1 | ACTA1 | actin, alpha skeletal muscle |
| 4 | 63055057 | NP_001017992.1 | ACTBL2 | beta-actin-like protein 2 |
| 5 | 12025678 | NP_004915.2 | ACTN4 | alpha-actinin-4 |
| 6 | 5031571 | NP_005713.1 | ACTR2 | actin-related protein 2 isoform b |
| 7 | 206597441 | NP_001128640.1 | ALDH3A1 | aldehyde dehydrogenase, dimeric |
| | | | | NADP-preferring |
| 8 | 34577112 | NP_908932.1 | ALDOA | fructose-bisphosphate aldolase A |
| | | | | isoform 1 |
| 9 | 4502067 | NP_001624.1 | AMBP | protein AMBP preproprotein |
| 10 | 10280622 | NP_066188.1 | AMY2B | alpha-amylase 2B precursor |
| 11 | 4502133 | NP_001630.1 | APCS | serum amyloid P-component |
| | | | | precursor |
| 12 | 346644849 | NP_001231178.1 | APEX1 | DNA-(apurinic or apyrimidinic site) |
| | | | | lyase |
| 13 | 4557321 | NP_000030.1 | APOA1 | apolipoprotein A-I preproprotein |
| 14 | 4502149 | NP_001634.1 | APOA2 | apolipoprotein A-II preproprotein |
| 15 | 153266841 | NP_000033.2 | APOH | beta-2-glycoprotein 1 precursor |
| 16 | 56676393 | NP_001166.3 | ARHGDIB | rho GDP-dissociation inhibitor 2 |
| 17 | 5031601 | NP_005711.1 | ARPC1B | actin-related protein 2/3 complex |
| | | | | subunit 1B |
| 18 | 146229327 | NP_001078897.1 | ARSA | arylsulfatase A isoform b |
| 19 | 157276599 | NP_001716.2 | BPI | bactericidal permeability-increasing |
| | | | | protein precursor |
| 20 | 169636415 | NP_000056.2 | C6 | complement component C6 |
| | | | | precursor |
| 21 | 4557395 | NP_000058.1 | CA2 | carbonic anhydrase 2 |
| 22 | 4885111 | NP_005176.1 | CALML3 | calmodulin-like protein 3 |
| 23 | 223278387 | NP_059118.2 | CALML5 | calmodulin-like protein 5 |
| 24 | 5453597 | NP_006126.1 | CAPZA1 | F-actin-capping protein subunit |
| | | | | alpha-1 |
| 25 | 4826659 | NP_004921.1 | CAPZB | F-actin-capping protein subunit beta |
| | | | | isoform 1 |
| 26 | 6912286 | NP_036246.1 | CASP14 | caspase-14 precursor |
| 27 | 4502693 | NP_001760.1 | CD9 | CD9 antigen |
| 28 | 68161541 | NP_001020083.1 | CEACAM1 | carcinoembryonic antigen-related |
| | | | | cell adhesion molecule 1 isoform 2 |
| | | | | precursor |
| 29 | 68508957 | NP_001257.4 | CES1 | liver carboxylesterase 1 isoform c |
| | | | | precursor |
| 30 | 67782358 | NP_001701.2 | CFB | complement factor B preproprotein |
| 31 | 62739186 | NP_000177.2 | CFH | complement factor H isoform a |
| | | | | precursor |
| 32 | 118442839 | NP_002104.2 | CFHR1 | complement factor H-related protein |
| | | | | 1 precursor |
| 33 | 5031695 | NP_005657.1 | CFHR2 | complement factor H-related protein |
| | | | | 2 precursor |
| 34 | 119392081 | NP_000195.2 | CFI | complement factor I preproprotein |
| 35 | 156627579 | NP_003269.2 | CLEC3B | tetranectin precursor |
| 36 | 14251209 | NP_001279.2 | CLIC1 | chloride intracellular channel |
| | | | | protein 1 |
| 37 | 110349772 | NP_000079.2 | COL1A1 | collagen alpha-1(I) chain |
| | | | | preproprotein |
| 38 | 4502951 | NP_000081.1 | COL3A1 | collagen alpha-1(III) chain |
| | | | | preproprotein |
| 39 | 89276751 | NP_000084.3 | COL5A1 | collagen alpha-1(V) chain |
| | | | | preproprotein |
| 40 | 4557485 | NP_000087.1 | CP | ceruloplasmin precursor |
| 41 | 4503009 | NP_001864.1 | CPE | carboxy peptidase E preproprotein |
| 42 | 153251270 | NP_060810.2 | CPPED1 | calcineurin-like phosphoesterase |
| | | | | domain-containing protein 1 |
| | | | | isoform a |
| 43 | 300244560 | NP_006052.2 | CRISP3 | cysteine-rich secretory protein 3 |
| | | | | isoform 1 precursor |
| 44 | 38327625 | NP_004068.2 | CS | citrate synthase, mitochondrial |
| | | | | precursor |
| 45 | 4885165 | NP_005204.1 | CSTA | cystatin-A |
| 46 | 189083844 | NP_001805.3 | CTSC | dipeptidyl peptidase 1 isoform a |
| | | | | preproprotein |
| 47 | 384081594 | NP_001244901.1 | CTSL1 | cathepsin L1 isoform 1 |
| | | | | preproprotein |
| 48 | 11128019 | NP_061820.1 | CYCS | cytochrome c |
| 49 | 116235485 | NP_620711.3 | DNER | delta and Notch-like epidermal |
| | | | | growth factor-related receptor |
| | | | | precursor |
| 50 | 62420888 | NP_037511.2 | DPP7 | dipeptidyl peptidase 2 preproprotein |
| 51 | 119703744 | NP_001933.2 | DSG1 | desmoglein-1 preproprotein |
| 52 | 58530842 | NP_001008844.1 | DSP | desmoplakin isoform II |
| 53 | 289063435 | NP_001165911.1 | ENDOU | poly(U)-specific endoribonuclease |
| | | | | isoform 3 precursor |
| 54 | 4503571 | NP_001419.1 | ENO1 | alpha-enolase isoform 1 |
| 55 | 94818891 | NP_001035548.1 | ERAP1 | endoplasmic reticulum |
| | | | | aminopeptidase 1 isoform b |
| | | | | precursor |
| 56 | 7657069 | NP_055399.1 | ERO1L | ERO1-like protein alpha precursor |
| | | NP_001156758.1 | EWSR1 | RNA-binding protein EWS isoform 4 |
| 58 | 4557581 | NP_001435.1 | FABP5 | fatty acid-binding protein, epidermal |
| | | | | low affinity immunoglobulin |
| 59 | 189083842 | NP_001121068.1 | FCGR3A | gamma Fc region receptor III-A |
| | | | | isoform d precursor |
| | | | | low affinity immunoglobulin |
| 60 | 132814489 | NP_000561.3 | FCGR3B | gamma Fc region receptor III-B |
| | | | | isoform 2 precursor |
| 61 | 70906435 | NP_005132.2 | FGB | fibrinogen beta chain isoform 1 |
| | | | | preproprotein |
| 62 | 70906437 | NP_000500.2 | FGG | fibrinogen gamma chain isoform |
| | | | | gamma-A precursor |
| 63 | 62122917 | NP_001014364.1 | FLG2 | filaggrin-2 |
| 64 | 108773793 | NP_001035810.1 | G6PD | glucose-6-phosphate 1- |
| | | | | dehydrogenase isoform b |
| 65 | 38202257 | NP_938148.1 | GANAB | neutral alpha-glucosidase AB |
| | | | | isoform 2 precursor |
| 66 | 378404908 | NP_001243728.1 | GAPDH | glyceraldehyde-3-phosphate |
| | | | | dehydrogenase isoform 2 |
| 67 | 32483410 | NP_000574.2 | GC | vitamin D-binding protein isoform 1 |
| | | | | precursor |
| 68 | 4504061 | NP_002067.1 | GNS | N-acetylglucosamine-6-sulfatase |
| | | | | precursor |
| 69 | 189083772 | NP_001121134.1 | GSN | gelsolin isoform b |
| 70 | 4504169 | NP_000169.1 | GSS | glutathione synthetase |
| 71 | 23065547 | NP_666533.1 | GSTM1 | glutathione S-transferase Mu 1 |
| | | | | isoform 2 |
| 72 | 4504183 | NP_000843.1 | GSTP1 | glutathione S-transferase P |
| 73 | 4504351 | NP_000510.1 | HBD | hemoglobin subunit delta |
| 74 | 7657603 | NP_055135.1 | HEBP2 | heme-binding protein 2 |
| 75 | 117190254 | NP_001070911.1 | HNRNPC | heterogeneous nuclear |
| | | | | ribonucleoproteins C1/C2 isoform b |
| 76 | 4826762 | NP_005134.1 | HP | haptoglobin isoform 1 preproprotein |
| 77 | 11321561 | NP_000604.1 | HPX | hemopexin precursor |
| 78 | 4504489 | NP_000403.1 | HRG | histidine-rich glycoprotein precursor |
| 79 | 20149594 | NP_031381.2 | HSP90AB1 | heat shock protein HSP 90-beta |
| 80 | 194248072 | NP_005336.3 | HSPA1A | heat shock 70 kDa protein 1A/1B |
| 81 | 27894321 | NP_776215.1 | IL1RN | interleukin-1 receptor antagonist |
| | | | | protein isoform 4 |
| 82 | 89191865 | NP_000202.2 | ITGB2 | integrin beta-2 precursor |
| 83 | 4504875 | NP_002248.1 | KLK1 | kallikrein-1 preproprotein |
| 84 | 209862865 | NP_001129504.1 | KLK11 | kallikrein-11 isoform 1 precursor |
| 85 | 4504893 | NP_000884.1 | KNG1 | kininogen-1 isoform 2 precursor |
| 86 | 119395750 | NP_006112.3 | KRT1 | keratin, type II cytoskeletal 1 |
| 87 | 5031839 | NP_005545.1 | KRT6A | keratin, type II cytoskeletal 6A |
| 88 | 119703753 | NP_005546.2 | KRT6B | keratin, type II cytoskeletal 6B |
| 89 | 55956899 | NP_000217.2 | KRT9 | keratin, type I cytoskeletal 9 |
| 90 | 56682962 | NP_005597.3 | LGMN | legumain preproprotein |
| 91 | 16418467 | NP_443204.1 | LRG1 | leucine-rich alpha-2-glycoprotein |
| | | | | precursor |
| 92 | 4505185 | NP_002406.1 | MIF | macrophage migration inhibitory |
| | | | | factor |
| 93 | 205277383 | NP_066278.3 | MST1 | hepatocyte growth factor-like |
| | | | | protein precursor |
| 94 | 310110100 | XP_003119529.1 | MUC5AC | PREDICTED: mucin-5AC |
| 95 | 12667788 | NP_002464.1 | MYH9 | myosin-9 |
| 96 | 66392203 | NP_001018146.1 | NME1- | NME1-NME2 protein |
| | | | NME2 | |
| 97 | 5453678 | NP_006423.1 | NPC2 | epididymal secretory protein E1 |
| | | | | precursor |
| 98 | 156564357 | NP_000895.2 | NQO2 | ribosyldihydronicotinamide |
| | | | | dehydrogenase [quinone] |
| 99 | 4826870 | NP_005004.1 | NUCB2 | nucleobindin-2 precursor |
| 100 | 5031985 | NP_005787.1 | NUTF2 | nuclear transport factor 2 |
| 101 | 167857790 | NP_000598.2 | ORM1 | alpha-1-acid glycoprotein 1 |
| | | | | precursor |
| 102 | 20070125 | NP_000909.2 | P4HB | protein disulfide-isomerase |
| | | | | precursor |
| 103 | 183227678 | NP_001116849.1 | PARK7 | protein DJ-1 |
| 104 | 4505621 | NP_002558.1 | PEBP1 | phosphatidylethanolamine-binding |
| | | | | protein 1 preproprotein |
| 105 | 4826898 | NP_005013.1 | PFN1 | profilin-1 |
| 106 | 50593010 | NP_000281.2 | PGAM2 | phosphoglycerate mutase 2 |
| 107 | 40068518 | NP_002622.2 | PGD | 6-phosphogluconate dehydrogenase, |
| | | | | decarboxylating |
| 108 | 4505763 | NP_000282.1 | PGK1 | phosphoglycerate kinase 1 |
| 109 | 6912586 | NP_036220.1 | PGLS | 6-phosphogluconol actonase |
| 110 | 4827036 | NP_005082.1 | PGLYRP1 | peptidoglycan recognition protein 1 |
| | | | | precursor |
| 111 | 4505821 | NP_002643.1 | PIP | prolactin-inducible protein precursor |
| 112 | 4505881 | NP_000292.1 | PLG | plasminogen isoform 1 precursor |
| 113 | 223718250 | NP_001138791.1 | PLS1 | plastin-1 |
| 114 | 288915539 | NP_001165806.1 | PLS3 | plastin-3 isoform 2 |
| 115 | 157168362 | NP_000261.2 | PNP | purine nucleoside phosphorylase |
| 116 | 19923106 | NP_000437.3 | PON1 | serum paraoxonase/arylesterase 1 |
| | | | | precursor |
| 117 | 209863034 | NP_001129408.1 | POSTN | periostin isoform 4 precursor |
| 118 | 10863927 | NP_066953.1 | PPIA | peptidyl-prolyl cis-trans isomerase |
| | | | | A |
| 119 | 4758950 | NP_000933.1 | PPIB | peptidyl-prolyl cis-trans isomerase |
| | | | | B precursor |
| 120 | 5453549 | NP_006397.1 | PRDX4 | peroxiredoxin-4 precursor |
| 121 | 341915348 | XP_003403487.1 | PRIM2 | PREDICTED: LOW QUALITY |
| | | | | PROTEIN: DNA primase large |
| | | | | subunit |
| 122 | 4506153 | NP_002764.1 | PRSS8 | prostasin preproprotein |
| 123 | 71361688 | NP_002768.3 | PRTN3 | myeloblastin precursor |
| 124 | 11386147 | NP_002769.1 | PSAP | proactivator polypeptide isoform a |
| | | | | preproprotein |
| 125 | 4506181 | NP_002778.1 | PSMA2 | proteasome subunit alpha type-2 |
| 126 | 4506203 | NP_002790.1 | PSMB7 | proteasome subunit beta type-7 |
| | | | | proprotein |
| 127 | 32171249 | NP_000945.3 | PTGDS | prostaglandin-H2 D-isomerase |
| | | | | precursor |
| 128 | 226056130 | NP_001139581.1 | PTGR1 | prostaglandin reductase 1 isoform 2 |
| 129 | 22035620 | NP_660183.1 | PYCARD | apoptosis-associated speck-like |
| | | | | protein containing a CARD isoform b |
| 130 | 255653002 | NP_001157412.1 | PYGL | glycogen phosphorylase, liver form |
| | | | | isoform 2 |
| 131 | 4506387 | NP_002865.1 | RAD23B | UV excision repair protein RAD23 |
| | | | | homolog B isoform 1 |
| 132 | 55743122 | NP_006735.2 | RBP4 | retinol-binding protein 4 precursor |
| 133 | 301129180 | NP_001180303.1 | RETN | resistin precursor |
| 134 | 45243507 | NP_002926.2 | RNASE3 | eosinophil cationic protein |
| | | | | precursor |
| 135 | 5032057 | NP_005611.1 | S100A11 | protein S100-A11 |
| 136 | 5032059 | NP_005612.1 | S100A12 | protein S100-A12 |
| 137 | 4506765 | NP_002952.1 | S100A4 | protein S100-A4 |
| 138 | 115298657 | NP_002954.2 | S100A7 | protein S100-A7 |
| 139 | 28827815 | NP_789793.1 | S100A7A | protein S100-A7A |
| 140 | 5174663 | NP_005971.1 | S100P | protein S100-P |
| 141 | 4507809 | NP_003348.1 | SCGB1A1 | uteroglobin precursor |
| 142 | 5729909 | NP_006542.1 | SCGB1D2 | secretoglobin family ID member 2 |
| | | | | precursor |
| 143 | 50363221 | NP_001002235.1 | SERPINA1 | alpha-1-antitrypsin precursor |
| 144 | 50659080 | NP_001076.2 | SERPINA3 | alpha-1-antichymotrypsin precursor |
| 145 | 205277441 | NP_000345.2 | SERPINA7 | thyroxine-binding globulin |
| | | | | precursor |
| 146 | 5902072 | NP_008850.1 | SERPINB3 | serpin B3 |
| 147 | 28076869 | NP_002965.1 | SERPINB4 | serpin B4 |
| 148 | 4502261 | NP_000479.1 | SERPINC1 | antithrombin-III precursor |
| 149 | 39725934 | NP_002606.3 | SERPINF1 | pigment epithelium-derived factor |
| | | | | precursor |
| 150 | 260064050 | NP_001159393.1 | SERPINF2 | alpha-2-antiplasmin isoform b |
| | | | | precursor |
| 151 | 73858568 | NP_000053.2 | SERPING1 | plasma protease C1 inhibitor |
| | | | | precursor |
| 152 | 5454052 | NP_006133.1 | SFN | 14-3-3 protein sigma |
| 153 | 4506925 | NP_003013.1 | SH3BGRL | SH3 domain-binding glutamic acid- |
| | | | | rich-like protein |
| 154 | 13775198 | NP_112576.1 | SH3BGRL3 | SH3 domain-binding glutamic acid- |
| | | | | rich-like protein 3 |
| 155 | 4507065 | NP_003055.1 | SLP1 | antileukoproteinase precursor |
| 156 | 67782309 | NP_001019637.1 | SOD2 | superoxide dismutase [Mn], |
| | | | | mitochondrial isoform B precursor |
| 157 | 118582275 | NP_003093.2 | SOD3 | extracellular superoxide dismutase |
| | | | | [Cu—Zn] precursor |
| 158 | 190341024 | NP_004675.3 | SPARCL1 | SPARC-like protein 1 precursor |
| 159 | 45827734 | NP_005978.2 | SPRR1A | cornifin-A |
| 160 | 5174693 | NP_005979.1 | SPRR2A | small proline-rich protein 2A |
| 161 | 62955831 | NP_001017418.1 | SPRR2B | small proline-rich protein 2B |
| 162 | 62945419 | NP_001014450.1 | SPRR2F | small proline-rich protein 2F |
| 163 | 281485608 | NP_003217.3 | TFF3 | trefoil factor 3 precursor |
| 164 | 189458821 | NP_003236.3 | TGM3 | protein-glutamine gamma- |
| | | | | glutamyltransferase E |
| 165 | 40317626 | NP_003237.2 | THBS1 | thrombospondin-1 precursor |
| 166 | 205277463 | NP_001128527.1 | TKT | transketolase isoform 1 |
| 167 | 4758508 | NP_004253.1 | TMPRSS11D | transmembrane protease serine 11D |
| 168 | 223555975 | NP_001138632.1 | TPM4 | tropomyosin alpha-4 chain isoform 1 |
| 169 | 4759270 | NP_004613.1 | TSN | translin |
| 170 | 21264578 | NP_005718.2 | TSPAN1 | tetraspanin-1 |
| 171 | 50592994 | NP_003320.2 | TXN | thioredoxin isoform 1 |
| 172 | 14249348 | NP_116120.1 | TXNDC17 | thioredoxin domain-containing |
| | | | | protein 17 |
| 173 | 166158922 | NP_001107227.1 | TYMP | thymidine phosphorylase isoform 1 |
| | | | | proprotein |
| 174 | 110611228 | NP_009055.2 | UTRN | utrophin |
| 175 | 4507869 | NP_003361.1 | VASP | vasodilator-stimulated |
| | | | | phosphoprotein |
| 176 | 88853069 | NP_000629.3 | VTN | vitronectin precursor |
| 177 | 5803225 | NP_006752.1 | YWHAE | 14-3-3 protein epsilon |
| 178 | 21735625 | NP_663723.1 | YWHAZ | 14-3-3 protein zeta/delta |
| 179 | 211058421 | NP_001129973.1 | ZNF844 | zinc finger protein 844 |

TABLE 2
93 proteins that are found as upregulated

| Sl No | Marker |
|---|---|
| 1 | S100A7 |
| 2 | COL1A1 |
| 3 | CD44 |
| 4 | APOA2 |
| 5 | S100A7A |
| 6 | S100A11 |
| 7 | SERPINA1 |
| 8 | AMBP |
| 9 | SERPINA7 |
| 10 | KRT6B |
| 11 | SLPI |
| 12 | APCS |
| 13 | POSTN |
| 14 | COL3A1 |
| 15 | S100P |
| 16 | GC |
| 17 | A1BG |
| 18 | HPX |
| 19 | LRG1 |
| 20 | KLK1 |
| 21 | CFB |
| 22 | APOH |
| 23 | CFH |
| 24 | VTN |
| 25 | THBS1 |
| 26 | RNASE3 |
| 27 | CEACAM1 |
| 28 | HP |
| 29 | ORM1 |
| 30 | PRTN3 |
| 31 | CP |
| 32 | APOA1 |
| 33 | HRG |
| 34 | SERPINC1 |
| 35 | FGB |
| 36 | PLG |
| 37 | PSAP |
| 38 | PRSS8 |
| 39 | SERPINA3 |
| 40 | CA2 |
| 41 | SERPINB4 |
| 42 | PYCARD |
| 43 | FGG |
| 44 | KLK11 |
| 45 | TYMP |
| 46 | PIP |
| 47 | PGLYRP1 |
| 48 | RBP4 |
| 49 | KNG1 |
| 50 | SERPINF1 |
| 51 | COL5A1 |
| 52 | HBD |
| 53 | CSTA |
| 54 | SPRR1A |
| 55 | SCGB1A1 |
| 56 | SPARCL1 |
| 57 | S100A12 |
| 58 | ITGB2 |
| 59 | CD9 |
| 60 | VASP |
| 61 | SERPINF2 |
| 62 | SERPING1 |
| 63 | MUC5AC |
| 64 | CFI |
| 65 | MST1 |
| 66 | C6 |
| 67 | DSP |
| 68 | KRT6A |
| 69 | NME1-NME2 |
| 70 | PLS3 |
| 71 | HNRNPC |
| 72 | EWSR1 |
| 73 | FCGR3B |
| 74 | TFF3 |
| 75 | CRISP3 |
| 76 | TSPAN1 |
| 77 | RETN |
| 78 | CLEC3B |
| 79 | RAD23B |
| 80 | SPRR2B |
| 81 | NPC2 |
| 82 | CFHR1 |
| 83 | PTGDS |
| 84 | PRDX4 |
| 85 | UTRN |
| 86 | SPRR2A |
| 87 | SPRR2F |
| 88 | SCGB1D2 |
| 89 | CFHR2 |
| 90 | CYCS |
| 91 | FCGR3A |
| 92 | PRIM2 |
| 93 | ZNF844 |

93 out of the total 179 differentials were found as upregulated (<1.5 fold) in all experiments or at least 2 out of the 3 experiments in at least one of the disease condition. These 93 proteins were scored as mentioned previously and based on its association with oral cancer (presence in OSCC database, Human protein atlas), biological relevance and its secretability (based on Exocarta, Signal P, and Secretome P analysis), 89/93 proteins were predicted as secretory by the prediction algorithms mentioned above. The major locus represented by the differentially expressed proteins in OSCC saliva was 1q21.3 (S100A11; S100A12; S100A7; S100A7A; SPRR2A; SPRR2B; SPRR2F; SPRR1A). These 93 proteins comprise a list of potential molecules that can be assessed for it clinical utility in OSCC saliva.

### Marker Validation:

Selected molecules from this list based on score and chromosomal location was further validated in a large cohort of patients and controls by using ELISA method. We verified 8 representative molecules (S100A7, CD44, COL5A1, COL1A1, S100P, S100A11, S100A15 and a1AT) by ELISA in independent cohorts of patients.

Saliva samples used were from four conditions; healthy controls, leukoplakia (pre-malignant), lymph node negative and lymph node positive OSCC patients. The samples were centrifuged at 10000 rpm to remove the cells and debris. ELISA was performed using commercially available kits from USCN, (Cloud-Clone Corp, US) or SUNREDBIO (Sunred biological technology Pvt Ltd, China) as per the manufacturer's instructions. ELISA for S100A7, COL1A1, S100P, S100A11, S100A15 and a1AT was performed using USCN kit and ELISA for CD44 and COL5A1 was performed using kit from SUNREDBIO. ELISA for CD44 was carried out in 20 samples from each of the four study groups. 11 samples from each of the 4 groups were used for ELISA for the other markers.

In the ELISA based assay we found that the mean concentration of the S100A7 in saliva was 264.17±86 pg/ml, 1360.9±288 pg/ml and 2324.4±259 pg/ml in normal (n=9), dysplastic leukoplakia (n=10) and oral cancer (n=18) respectively (FIG. 5D). Thus, the levels of S100 A7 were significantly increased in the saliva as the disease progresses from premalignant stage to cancer. Further subgroup analysis of the patients diagnosed with cancer indicated that the mean concentration of S100A7 was 1948.5 pg/ml (SE 404) and 2662.67 pg/ml (SE 303) respectively in lymph node negative and lymph node positive oral cancers respectively.

The mean concentration of CD44 protein in saliva was 110.02±16 ng/ml, 188.45±15 ng/ml and 243.18±11 ng/ml in normal (n=20), dysplastic leukoplakia (n=20) and oral cancer (n=40) respectively (FIG. 5A). The mean concentration of CD44 was 220.89±15 ng/ml and 265.47±15 ng/ml respectively in lymph node negative and lymph node positive oral cancers respectively. Thus, the levels of CD44 were increased in the saliva as the disease progresses from premalignant stage to cancer.

The mean concentration of COL1A1 protein in saliva was 176.84 ±63.18 pg/ml, 438.45 ±178 pg/ml and 774.96 ±81 pg/ml in normal (n=8), dysplastic leukoplakia (n=8) and oral cancer (n=16) respectively. Further, COL1A1 levels were significantly elevated in lymph node positive compared to lymph node negative. The mean concentration of COL1A1 was 575.2±72 pg/ml and 974.67±107 pg/ml respectively in lymph node negative and lymph node positive oral cancers respectively.

The mean concentration of COL5A1 protein in saliva was 62.83±4.6 ng/ml, 89.02±5.8 ng/ml and 109.83±3.8 ng/ml in normal (n=6), Dysplastic leukoplakia (n=7) and Oral cancer (n=17) respectively (FIG. 5B). The mean concentration of COL5A1 was 106.19±4.3 ng/ml and 113.93±6.5 ng/ml respectively in lymph node negative (n=9) and lymph node positive oral cancers (n=8) respectively.

The results from ELISA showed that the mean concentration of S100P protein in saliva was 0.84±0.237 ng/ml, 2.59±0.68 ng/ml and 3.33±0.50 ng/ml in normal (n=11), dysplastic leukoplakia (n=11) and oral cancer (n=22) respectively (FIG. 5C). The mean concentration of S100P was 3.10±0.50 ng/ml and 3.57±0.51 ng/ml respectively in lymph node negative and lymph node positive oral cancers respectively. Thus, the levels of S100P were increased in the saliva as the disease progresses from normal to the premalignant stage to cancer. The p values based on t-test were significant for normal Vs leukoplakia (p=0.016) normal Vs tumor (p=1.00034E-06), normal vs lymph node negative/positive tumors (p=0.000605749, 0.000138422). This suggests that S100P can be used as a salivary marker to identify early stage leukoplakia and cancer.

The mean concentration of S100A11 protein in saliva was 3118.4±808.8 pg/ml, 3788.9 ±613.28 pg/ml and 5592.9±303.62 pg/ml in normal (n=11), dysplastic leukoplakia (n=11) and oral cancer (n=22) respectively. The mean concentration of S100A11 was 5095.8±302 pg/ml and 6089.9±228 pg/ml respectively in lymph node negative and lymph node positive oral cancers respectively.

The results from ELISA showed that the mean concentration of S100A15 protein in saliva was 2±1.1 ng/ml, 2.06±0.7 ng/ml and 6.51±2.1 ng/ml in normal (n=11), dysplastic leukoplakia (n=11) and oral cancer (n=22) respectively . The mean concentration of S100A15 was 4.82±2.1 ng/ml and 8.2±2.2 ng/ml respectively in lymph node negative and lymph node positive oral cancers respectively. Thus, the levels of S100A15 were increased in the saliva as the disease progresses from premalignant stage to cancer.

Thus, results from ELISA showed that the mean concentration of A1AT protein in saliva was 2965.5±378 ng/ml, 3696.5±384 ng/ml and 4117.5±198 ng/ml in normal (n=11), dysplastic leukoplakia (n=11) and oral cancer (n=22) respectively. The mean concentration of A1AT was 3863.4±198 ng/ml and 4371.6±174 ng/ml respectively in lymph node negative and lymph node positive oral cancers respectively. Thus, the levels of A1AT were increased in the saliva as the disease progresses from premalignant stage to cancer.

Thus, out of all 8 markers validated by ELISA, inventors have 4 early diagnostic markers—S100A7, CD44, COL5A1, and S100P. Their levels were high in early stage leukoplakia and tumor. There are 4 markers for lymph node-negative vs positive—S100A11, a1AT, COL1A1, CD44 and can be used for the prognosis of patients. There is one marker which differentiates cancer from normal i.e., S100A15.

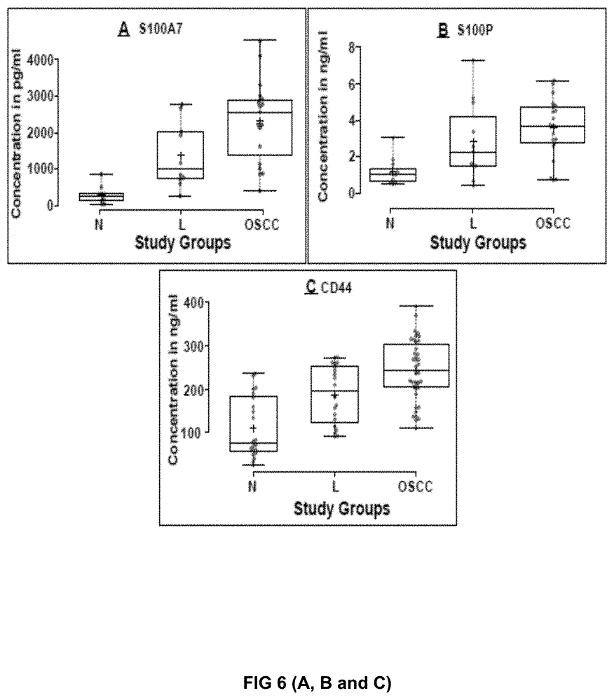

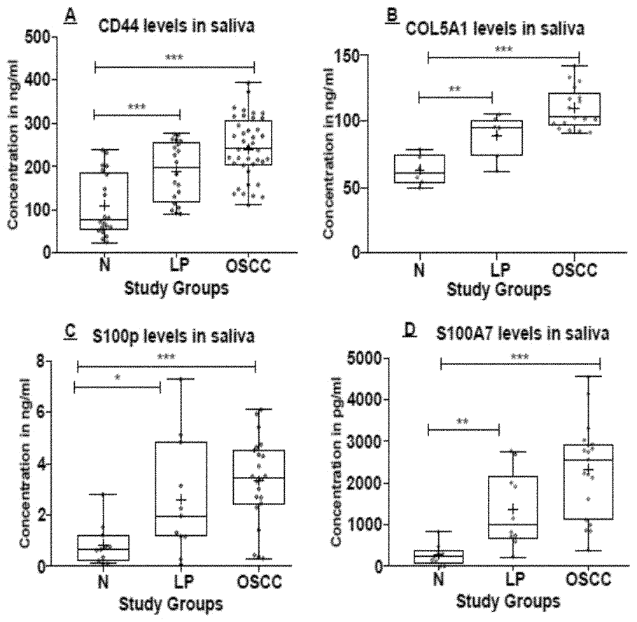

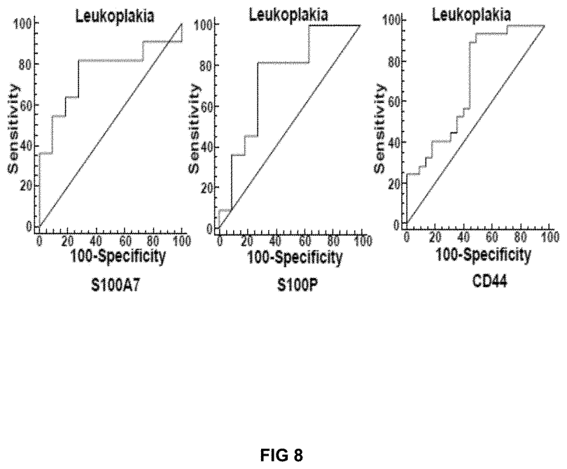

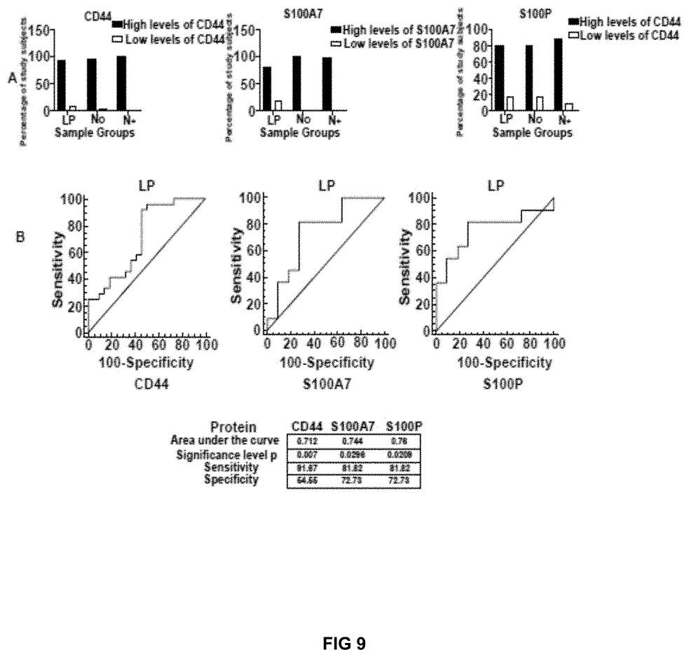

FIG. 6 (E, F, G and H) illustrates the COL1A1, a1AT, S100A11, S100A15 protein levels in the saliva of premalignant and malignant lesions of the oral cavity. Box plots indicate the average concentrations of COL1A1 (A), a1AT (B), S100A11 (C) and S100A15 (D) in the saliva of dysplastic leukoplakia and OSCC. The oral cancer samples include lymph node negative and lymph node positive OSCC.

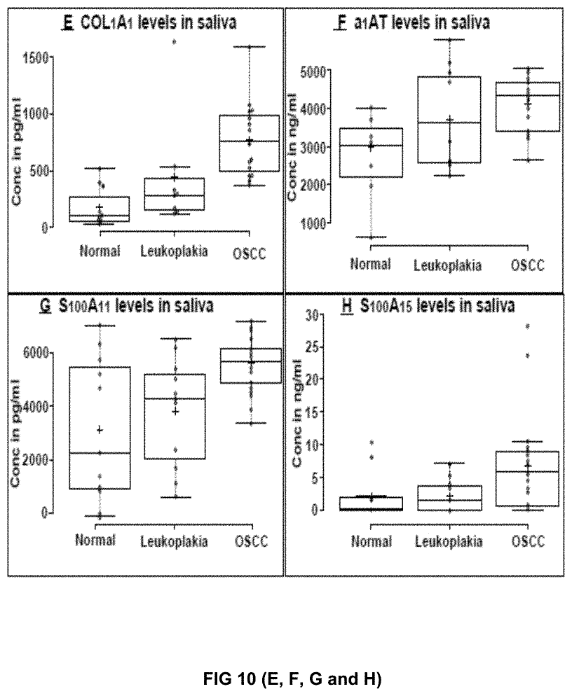

ROC curve analysis was carried out to test the diagnostic efficiency of these 4 potential markers. Three markers, CD44, S100A7 and S100P significantly differentiated leukoplakia based on the above analysis (FIG. 7).

TABLE 3

| | Area under the | Significance | | |
|---|---|---|---|---|
| Gene | ROC curve (AUC) | level P | Sensitivity | Specificity |
| S100A7 | 0.744 | 0.0296 | 81.82 | 72.73 |
| S100P | 0.76 | 0.0209 | 81.82 | 72.73 |
| CD44 | 0.712 | 0.007 | 91.67 | 54.55 |

Table 3, lists out the details of the ROC analysis of the three markers CD44, S100P and S100 A7 Salivary markers for the detection of Oral premalignant lesions and cancers.

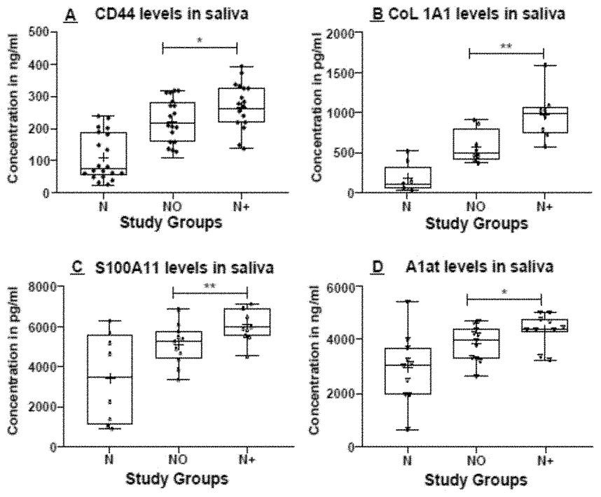

Sensitivity and specificity were 81.82% and 72.73% for S100A7, 81.82% and 72.73% for S100P, 91.67% and 54.55% for CD44 respectively implying that these markers can be utilized to make a potential non-invasive screening tool for oral leukoplakia. Since the concentrations of S100A7 and CD44 which are potential leukoplakia screening markers were significantly higher in tumor, we propose that these two markers may indicate highly potential markers for leukoplakia to cancer progression. FIG. 7 illustrate the significance of the three markers (7A) CD44, (7B) S100P and (7C) S100 A7 in early detection of oral premalignant disorders in the saliva of Normal, leukoplakia and OSCC patients. Box plots indicate the mean concentrations in each cohort and the ROC curve analysis indicates the specificity and sensitivity of the markers. FIG. 8 depicts early detection markers: ROC curves for S100A7, S100P and CD44.

Further ROC analysis showed that 6 (CD44, S100A7, S100P, S100A11, S100A15, SERPINA1) out of the 8 markers were significantly differentiating cancer from normal.

TABLE 4

| | Area under the | Significance | | |
|---|---|---|---|---|
| Gene | ROC curve (AUC) | level P | Sensitivity | Specificity |
| S100A7 | 0.893 | <0.0001 | 86.36 | 81.82 |
| S100P | 0.884 | <0.0001 | 81.82 | 90.91 |
| CD44 | 0.836 | <0.0001 | 69.77 | 81.82 |
| SERPINA1 | 0.826 | 0.0006 | 95.45 | 63.64 |
| S100A15 | 0.758 | 0.0075 | 86.36 | 63.64 |
| S100A11 | 0.756 | 0.0186 | 100 | 54.55 |

Table 4, Lists out the ROC details of the 6 markers that are efficient in detecting cancers salivary markers for oral cancers.

Sensitivity and specificity for each of the markers are provided in Table 4. The concentration of 4 markers (CD44, COL1A1, S100A11 and SERPINA1) were significantly high in lymph node positive cases compared to lymph node-negative cases and 3 (CD44, S100A11, and SERPINA1) of these 4 markers were supported by ROC analysis and can be used to differentiate lymph node positive tumors.

According to a non limiting exemplary aspect of the present invention, the primary advantage of the current invention is the non-invasive use of saliva as a medium of diagnosis/prognosis. The biomarkers identified in the study are predicted to identify dysplastic lesions and hence thereby can avoid unwanted biopsy. Additionally, the use of these markers will increase the accuracy of the diagnosis since these changes are highly specific.

According to a non limiting exemplary aspect of the present invention, these markers also have the probability of being used to assess the progression/regression of oral cancer after treatment. Since the molecular changes precede the histological and clinical changes, the use of this method can also facilitate early detection/prognosis.

According to one of the embodiments of the present invention, the present methodology compares salivary marker-based diagnosis tow biopsy towards detection of dysplastic lesions in the leukoplakia of the oral cavity. This study employed proteomic profiling to identify the candidate biomarkers and then further validated a selected subset by ELISA.

Compositions, Kits and Integrated Systems; the invention provides compositions, kits and integrated systems for practicing the assays/methods described herein using polynucleotides and/or polypeptides of the invention, antibodies specific for polypeptides or polynucleotides of the invention, etc. The kits typically include a probe that comprises an antibody that specifically binds to a specific polypeptides or polynucleotides of the invention, and a label for detecting the presence of the probe. In addition, the kits may include several antibodies specific for, or polynucleotide sequences encoding, the polypeptides of the invention.

Major advantage of the current invention is non-invasive method of detection.
Detection is based on molecular biomarkers which are more specific for premalignant lesions and cancer and hence can accurately predict or diagnose the disease.
Can be used to develop a point of care system that can enable high throughput screening in a community level setting.
Enables periodic monitoring of advanced stage patients with high risk of developing metastasis.

The possible uses of this invention include but not limited to,
Individual or combination of markers can be used to develop assay systems for the early diagnosis of high risk lesions
Individual or combination of markers can be used for community-based screening of high-risk populations to identify patients at risk for developing oral premalignant lesion and/or cancer
Individual or combination of markers can be used to develop monitoring or surveillance systems to monitor disease progression
Individual or combination of markers can be used to predict development of nodal metastasis
The marker panel can be used for the development of a Point-of-Care assay system that can be applied towards early detection, screening and disease progression.
These assay systems can be used for patients susceptible to or diagnosed with oral cancer and the subsites of head and neck cancer such as cancers of the oral cavity, pharynx, and larynx.

Merely for illustration, only representative number/type of graph, chart, block, and sub-block diagrams were shown. Many environments often contain many more block and sub-block diagrams or systems and sub-systems, both in number and type, depending on the purpose for which the environment is designed.

According to a non limiting exemplary aspect of the present invention, the markers can be used for the development of kits that enable saliva collection, processing, and marker detection. These diagnostic kits developed can then be utilized by hospitals/private clinics/dental doctors or the public as such to screen/diagnose oral cancer.

While specific embodiments of the invention have been shown and described in detail to illustrate the inventive principles, it will be understood that the invention may be embodied otherwise without departing from such principles.

Reference throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment” and similar language throughout this specification may, but do not necessarily, all refer to the same embodiment.

It should be understood that the figures and/or screen shots illustrated in the attachments highlighting the functionality and advantages of the present invention are presented for example purposes only. The present invention is sufficiently flexible and configurable, such that it may be utilized in ways other than that shown in the accompanying figures.

It should be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Coelho, K. R., Challenges of the oral cancer burden in India. J Cancer Epidemiol, 2012. 2012: p. 701932.
2. Daftary, D. K., Temporal role of tobacco in oral carcinogenesis: a hypothesis for the need to prioritize on precancer. Indian J Cancer, 2010. 47 Suppl 1: p. 105-7.
3. Loree, T. R. and E. W. Strong, Significance of positive margins in oral cavity squamous carcinoma. Am J Surg, 1990. 160(4): p. 410-4.
4. Cho, W. C., Proteomics and translational medicine: molecular biomarkers for cancer diagnosis, prognosis, and prediction of therapy outcome. Expert Rev Proteomics, 2011. 8(1): p. 1-4.
5. Pfaffe, T., et al., Diagnostic potential of saliva: current state and future applications. Clin Chem, 2011. 57(5): p. 675-87.
6. Shintani, S., et al., Identification of a truncated cystatin SA-I as a saliva biomarker for oral squamous cell carcinoma using the SELDI ProteinChip platform. Int J Oral Maxillofac Surg, 2010. 39(1): p. 68-74.
7. Zimmermann, B. G. and D. T. Wong, Salivary mRNA targets for cancer diagnostics. Oral Oncol, 2008. 44(5): p. 425-9.
8. Zhang, L., et al., Development of transcriptomic biomarker signature in human saliva to detect lung cancer. Cell Mol Life Sci, 2012. 69(19): p. 3341-50.
9. Messadi, D. V., Diagnostic aids for detection of oral precancerous conditions. Int J Oral Sci, 2013. 5(2): p. 59-65.

1. Biomarkers for carcinoma detection/diagnosis, wherein a combination of a plurality of biomarkers selected from Table 1 comprising 179 differentially regulated proteins or Table 2 comprising 93 differentially regulated proteins existing in body fluid of a subject.

2. The biomarker for carcinoma detection/diagnosis as claimed in claim 1, wherein the said biomarkers are salivary biomarkers.

3. The biomarker for carcinoma detection/diagnosis as claimed in claim 1, wherein said carcinoma/cancer is head and neck squamous cell carcinoma (HNSCC); oral carcinomas: cancers of larynx, pharynx; primary or secondary cancers.

4. The biomarker for carcinoma detection/diagnosis as claimed in claim 1, wherein the said body fluid is plasma, serum, urine, peripheral blood, sputum, saliva, or mucosal secretion.

5. The biomarkers as claimed in claim 1, wherein the biomarker is selected from the group consisting of S100A7, CD44, COL5A1, S100P COL1A1, CD44, S100A11, a1AT or any combinations thereof.

6. A method of identifying, diagnosing or providing a prognosis of oral cancer and pre-cancer in a biological sample from a subject, wherein the method comprising acts of:
(a) detecting the presence or level of one or more biomarker in a biological sample from a subject, wherein one or more biomarker are selected from Table 1 comprising 179 differentially regulated proteins or Table 2 comprising 93 differentially regulated proteins in a biological sample from an individual; and
(b) identifying/ determining whether or not the said biomarker is differentially expressed in the sample, thereby diagnosing or providing a prognosis for oral cancer and pre-cancer.

7. The method of identifying, diagnosing or providing a prognosis of oral cancer and pre-cancer in a subject as claimed in claim 6, wherein the presence of one or more of the biomarkers in a subject sample and correlating the levels of biomarkers with increased risk of developing oral carcinomas, subjecting samples to comparative evaluation with the levels of such biomarker(s) in normal subjects, wherein the preferred biomarker is selected from the group consisting of S100A7, CD44, COL5A1, S100P COL1A1, CD44.

8. The method of identifying, diagnosing or providing a prognosis of oral cancer and pre-cancer as claimed in claim 6 and claim 7, wherein the method comprises; comparing the levels of one or more biomarker in a biological sample from a subject with those present in a normal subject; evaluating the levels of such biomarkers; and indicating different stages of carcinoma/cancer such as pre, early or advanced carcinomas.

9. The method of identifying, diagnosing or providing a prognosis of oral cancer and pre-cancer as claimed in claim 6, wherein the method comprises determining the level of at least one of the set of oral cancer biomarkers by a method selected from the group consisting of an antibody based assay, ELISA, western blotting, targeted mass spectrometry, custom micro array and/or protein microarray, flow cytometry, immunofluorescence, PCR, immunohistochemistry, and a multiplex detection assay.

10. The method of identifying, diagnosing or providing a prognosis of oral cancer as claimed in claim 6, wherein the subject is a human being.

11. The method of identifying, diagnosing or providing a prognosis of oral cancer as claimed in claim 6, wherein the method is for screening a large or small group of subjects for prognosis, screening at various stages of cancers, including post-treatment surveillance.

12. The method of identifying, diagnosing or providing a prognosis of oral cancer as claimed in claim 6, wherein the method is for detecting risk of cancer development in subjects with tobacco or/and alcohol habit history, premalignant lesions, leukoplakia with high level of dysplasia, differentiating lymph node negative tumor from lymph node positive tumors; detecting salivary biomarkers for use in early diagnosis of dysplastic lesions/cancers of the oral cavity.

13. The biomarkers as claimed in claim 1 and claim 6, wherein the biomarkers are for non-invasive, early detection/prognosis method comprising protein profiling of saliva at different stages of oral cancer progression, surveillance/monitoring of the progression of disease in patients with HNSCC, periodic indicators of carcinogenesis susceptibility in patients with early premalignant lesions; and
wherein these biomarkers are combined with clinical and pathological parameters to improve efficacy of diagnosis/prognosis.

13. A kit for the diagnosis or prognosis of an oral cancer and/or the biomarker for the pathology disease or disorder, the biomarker selected from a combination of a plurality of biomarkers selected from Table 1 comprising 179 differentially regulated proteins or Table 2 comprising 93 differentially regulated proteins existing in body fluid of a subject, wherein the preferred biomarker is selected from the group consisting of S100A7, CD44, COL5A1, S100P COL1A1, CD44, S100A11, a1AT or any combinations thereof; and
b) a detector for the identifier;
wherein the identifier is associated to the biomarker in the bodily fluid, and the detector is used to detect the identifier, the identifier and the detector thereby enabling the detection of biomarker profile in the bodily fluid of a subject.

15. The kit as claimed in claim 14, wherein the kit is for oral pre-cancer lesions (leukoplakia) and/or diagnosing or providing a prognosis for oral cancer in a biological sample from a subject, the kit comprising of reagents that enable collection, processing and specific binding of cancer biomarkers.

16. The kit as claimed in claim 14, wherein at least one oral cancer biomarker is selected from Table 1 or Table 2.

17. An antibody generated against an epitope selected from the group consisting of those found in Tables 1 and Table 2 of claim 1 and claim 14.

18. A kit for use in diagnosing or providing a prognosis for oral cancer, the kit comprising an antibody of claim 15.
